(19) United States
(12) Patent Application Publication
(10) Pub. No.: US 2011/0046245 A1
(43) Pub. Date: Feb. 24, 2011

(54) Catalytic Reaction Module

(75) Inventors: Michael Joseph Bowe, Lancashire (GB); Clive Derek Lee-Tuffnell, Oxfordshire (GB); Robert Peat, Oxfordshire (GB)

Correspondence Address: MCDERMOTT WILL & EMERY LLP, 600 13TH STREET, N.W., WASHINGTON, DC, 20005-3096, US

(73) Assignee: CompactGTL plc, Oxfordshire (GB)

(21) Appl. No.: 12/865,974

(22) PCT Filed: Feb. 10, 2009
(86) PCT No.: PCT/GB2009/050129

§ 371 Date: Sep. 28, 2010

(30) Foreign Application Priority Data
Feb. 14, 2008 (GB) 0802726.0
Nov. 6, 2008 (GB) 0820281.4

(51) Int. Cl. B01J 8/02; B01J 19/00; C07C 1/04

(52) U.S. Cl. 518/712; 422/211; 422/198

(57) ABSTRACT

A catalytic reaction module (10) for performing an endothermic reaction such as steam methane reforming, includes separate reactor blocks (12), each reactor block defining a multiplicity of first and second flow channels (15, 16) arranged alternately within the block to ensure thermal contact between the first and second flow channels. The reactor blocks (12a, 12b) may be arranged and connected for series flow of a combustible gas mixture in the first flow channels (15) and also of a gas mixture to undergo the endothermic reaction in the second flow channels (16). This enables the combustion process to be carried out in stages, with the option of cooling the combustion gases between stages, and introducing additional fuel and additional air.

This invention relates to a catalytic reaction module with channels for performing an endothermic chemical reaction such as steam reforming, in which the heat is provided by a combustion reaction in adjacent channels, and to a method for performing an endothermic chemical reaction with such a module, and to the control of such a module.

A plant and process are described in WO 2005/102511 (GTL Microsystems AG) in which methane is reacted with steam, to generate carbon monoxide and hydrogen in a first catalytic reactor; the resulting gas mixture is then used to perform Fischer-Tropsch synthesis in a second catalytic reactor. The reforming reaction is typically carried out at a temperature of about 800° C., and the heat required may be provided by catalytic combustion in channels adjacent to those in which reforming is carried out, the combustion channels containing a catalyst which may comprise palladium or palladium/platinum on an alumina support in the form of a thin coating on a metallic substrate. An inflammable gas mixture such as a mixture of methane and air is supplied to the combustion channels. Combustion occurs at the surface of the catalyst without a flame. However, it has been found that the combustion reaction tends to occur most vigorously near the start of the combustion channel, which can lead to an unsuitable temperature distribution along the channel; although this problem may be overcome by staging fuel injection along the combustion channel, an alternative solution would be desirable.

According to the present invention there is provided a catalytic reaction module for performing an endothermic reaction, the module comprising a plurality of separate reactor blocks, each reactor block defining a multiplicity of first and second flow channels arranged alternately within the block to ensure thermal contact between the first and second flow channels, the reactor blocks being arranged and connected for series flow of a gas mixture to undergo the endothermic reaction in the first flow channels and also for flow of a combustible gas mixture in the second flow channels, such that the endothermic reaction mixture flows in series through the reactor blocks.

The reactor blocks are referred to as being separate in the sense that they have distinct and separate inlets and outlets for the gas mixtures. The reactor blocks may also be physically separate, that is to say spaced apart from each other; or they may be joined together for example as a stack.

Preferably the module is arranged such that the combustible gas mixture provided to a reactor block is at an elevated temperature below its auto-ignition temperature, the temperature being raised at least in part as a result of combustion of combustible gas mixture in one or more of the reactor blocks. Indeed preferably the combustible gas mixture provided to each reactor block in the module is at such an elevated temperature. For at least some of the blocks the temperature may be raised by heat exchange with gases emerging from the second gas flow channels of one or more of the reactor blocks. In one preferred embodiment the combustible gas mixture is arranged to flow in series through the reactor blocks in the same order as the endothermic gas mixture. In this case the combustible gas mixture provided to a second or subsequent reactor block is at an elevated temperature as a result of having at least partly undergone combustion in the preceding reactor block of the series.

The combustible gas mixture comprises a fuel (such as methane) and a source of oxygen (such as air). Preferably between successive reactor blocks means are provided to treat the outflowing gas mixture that has undergone combustion, for example to change its temperature, or to introduce and mix in additional fuel. It may also be desirable between successive reactor blocks to provide means to introduce additional air into the outflowing gas mixture that results from combustion. By staging the provision of fuel between different reactor blocks and by staging the introduction of air, greater control over the temperature distribution can be achieved. For example, if there are two reactor blocks in series, the proportion of the fuel provided at the first stage is preferably between 50% and 70% of the total required fuel, the remainder being provided for the second stage.

The invention also provides a method of performing an endothermic reaction wherein the heat required for the endothermic reaction is provided by a combustion reaction in an adjacent channel to the endothermic reaction, wherein the endothermic reaction is carried out in a plurality of successive stages. The endothermic reaction may be steam methane reforming, and in this case preferably the temperature in the endothermic reaction channels increases through the first stage to between 675° C. and 700° C., preferably to about 690° C.; and increases through the second stage to between 730° C. and 800° C., preferably to about 760° C. In a preferred embodiment the combustion reaction is also carried out in at least two successive stages, with treatment of the combustion gas mixture emerging from one stage before it is introduced to the next stage.

The treatment of the combustion gas mixture between successive stages preferably comprises changing its temperature and adding additional fuel. By lowering the gas temperature before adding additional fuel, auto-ignition can be avoided.

By performing the combustion process in a number of stages, using separate reactor blocks, the benefits of staged fuel injection are obtained—for example a more uniform temperature distribution along the reactor module—while avoiding potential problems. In particular this makes it possible to cool the combustion gas mixture between successive stages, before introducing additional fuel, which can ensure that auto-ignition does not occur. The treatment of the combustion gas mixture between successive reactor blocks takes place within the module, but not within the reactor blocks.

Preferably the first flow channels and the second flow channels extend in parallel directions, within a reactor block, and the combustible gas mixture and the endothermic reaction mixture flow in the same direction (co-flow). Preferably the flow channels are of length at least 300 mm, more preferably at least 500 mm, but preferably no longer than 1000 mm. A preferred length is between 500 mm and 700 mm, for example 600 mm. It has been found that co-flow operation gives better temperature control, and less risk of hot-spots.

In the preferred embodiment each first flow channel (the channels for the endothermic reaction) and each second flow channel (the channels for the combustion reaction) contains a removable catalyst structure to catalyse the respective reaction, each catalyst structure preferably comprising a metal substrate, and incorporating an appropriate catalytic material. Preferably each such catalyst structure is shaped so as to subdivide the flow channel into a multiplicity of parallel flow sub-channels. Preferably each catalyst structure includes a ceramic support material on the metal substrate, which provides a support for the catalyst.

The metal substrate provides strength to the catalyst structure and enhances thermal transfer by conduction. Preferably the metal substrate is of a steel alloy that forms an adherent surface coating of aluminium oxide when heated, for example a ferritic steel alloy that incorporates aluminium (eg Fecralloy™). The substrate may be a foil, a wire mesh or a felt sheet, which may be corrugated, dimpled or pleated; the preferred substrate is a thin metal foil for example of thickness less than 100 μm, which is corrugated to define the longitudinal sub-channels.

Each reactor block may comprise a stack of plates. For example, the first and second flow channels may be defined by grooves in respective plates, the plates being stacked and then bonded together. Alternatively the flow channels may be defined by thin metal sheets that are castellated and stacked alternately with flat sheets; the edges of the flow channels may be defined by sealing strips. To ensure the required good thermal contact both the first and the second gas flow channels may be between 10 mm and 2 mm high (in cross-section); and each channel may be of width between about 3 mm and 25 mm. The stack of plates forming the reactor block is bonded together for example by diffusion bonding, brazing, or hot isostatic pressing.

Preferably a flame arrestor is provided at the inlet to each flow channel for combustion to ensure a flame cannot propagate back into the combustible gas mixture being fed to the combustion channel. This may be within an inlet part of each combustion channel, for example in the form of a non-catalytic insert that subdivides a portion of the combustion channel adjacent to the inlet into a multiplicity of narrow flow paths which are no wider than the maximum gap size for preventing flame propagation. For example such a non-catalytic insert may be a longitudinally-corrugated foil or a plurality of longitudinally-corrugated foils in a stack. Alternatively or additionally, where the combustible gas is supplied through a header, then such a flame arrestor may be provided within the header.

The present invention also provides a method of performing an endothermic reaction, such as steam reforming, using such a reaction module. By combining air with the outflowing gas mixture that results from combustion, before adding additional fuel, the temperature of the combustible mixture can be held below the auto-ignition temperature, so ensuring that combustion occurs as a heterogeneous reaction at the surface of the catalyst structure (rather than occurring in the gas phase).

Performing steam methane reforming in this way enables operation to be carried out at a high space velocity within each block, for example between 10 000 and 60,000/hr, while attaining more than 90% of equilibrium conversion. Similarly the combustion reaction is preferably carried out at a space velocity between 20 000 and 70,000/hr. The space velocity, in this document, means the volume of gas supplied to a reactor per hour, measured at standard temperature and pressure (0° C. and 1 atmosphere), as a multiple of the free volume of the corresponding reactor channels.

The invention also provides a method of controlling combustion; and it provides a method of minimising thermal stresses in a compact catalytic reactor.

The invention will now be further and more particularly described, by way of example only, and with reference to the accompanying drawings, in which:

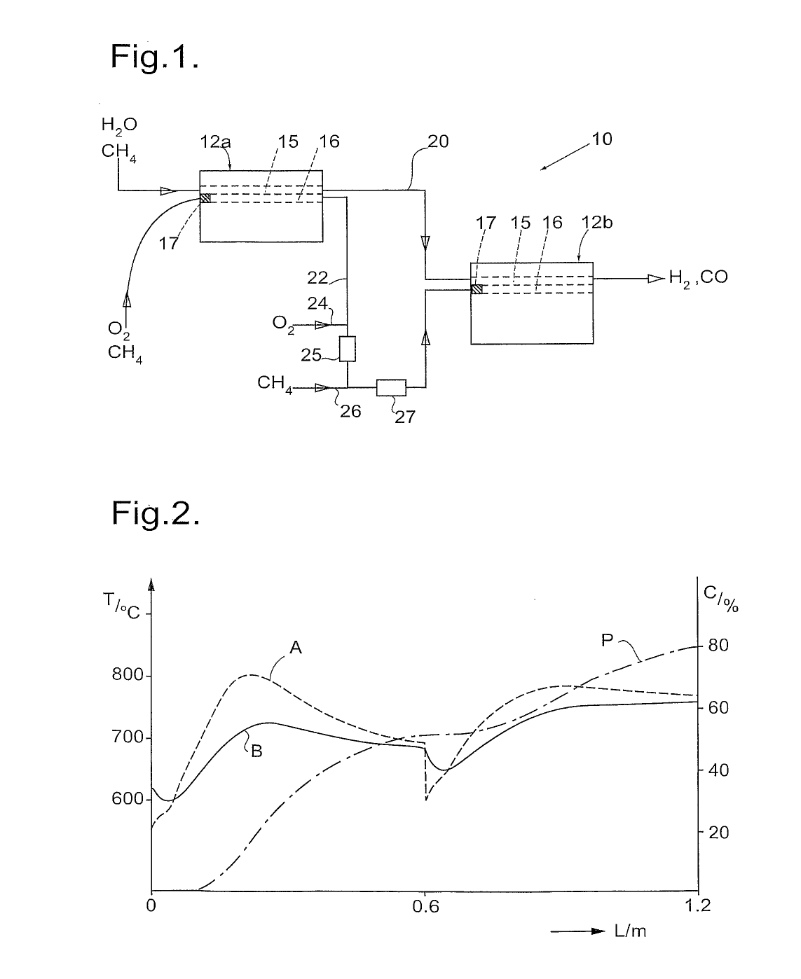

FIG. 1 shows a diagrammatic side view of a reaction module of the invention;

FIG. 2 shows graphically the variation of temperature through the reactor module of FIG. 1, and the corresponding variation of conversion in the steam methane reaction;

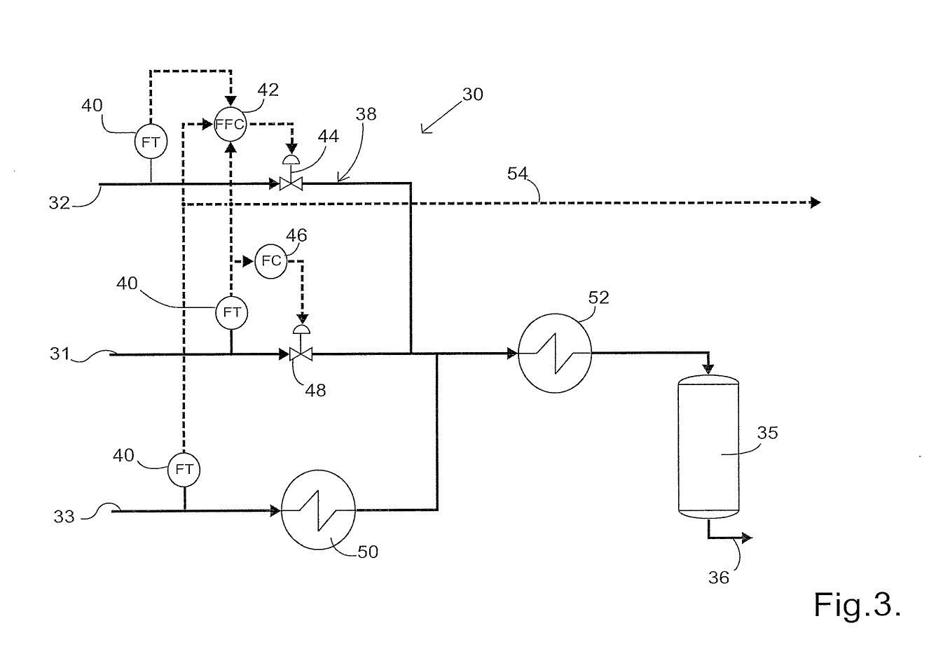

FIG. 3 shows a system whereby a steam methane mixture is supplied to the module of FIG. 1;

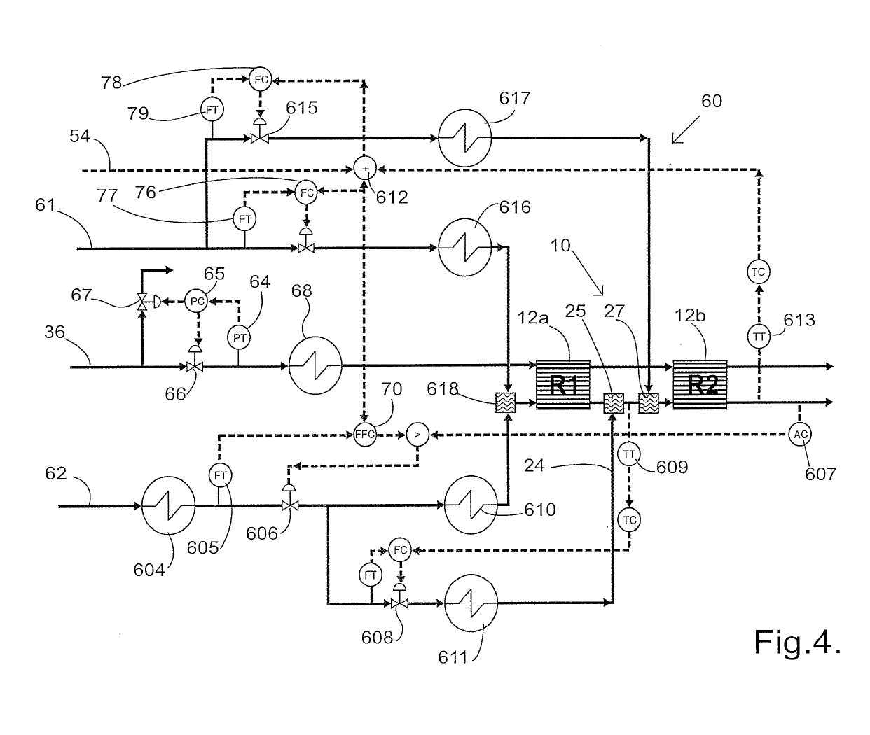

FIG. 4 shows a system that incorporates a module of FIG. 1; and

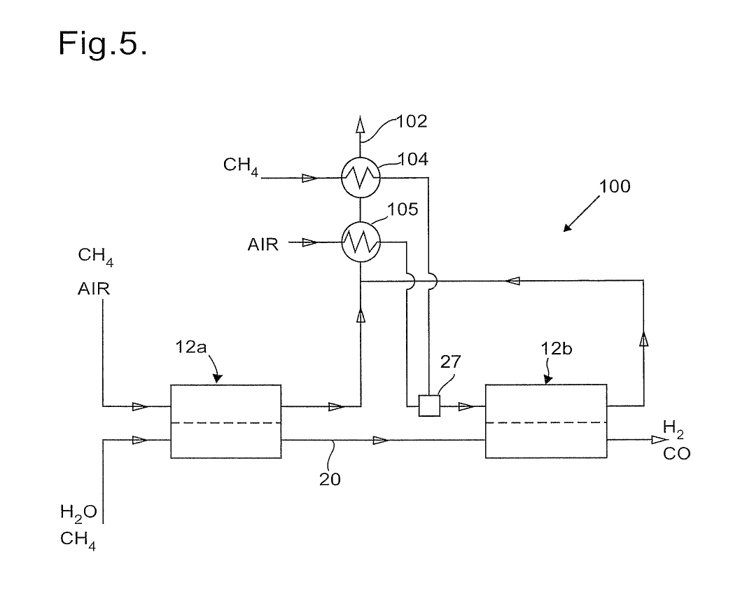

FIG. 5 shows a flow diagram of an alternative reaction module of the invention.

The steam reforming reaction of methane is brought about by mixing steam and methane, and contacting the mixture with a suitable catalyst at an elevated temperature so the steam and methane react to form carbon monoxide and hydrogen (which may be referred to as synthesis gas or syngas). The steam reforming reaction is endothermic, and the heat is provided by catalytic combustion, for example of methane mixed with air. The combustion takes place over a combustion catalyst within adjacent flow channels within a reforming reactor. Preferably the steam/methane mixture is preheated, for example to over 600° C., before being introduced into the reactor. The temperature in the reformer reactor therefore typically increases from about 600° C. at the inlet to about 750-800° C. at the outlet.

The total quantity of fuel (e.g. methane) that is required is that needed to provide the heat for the endothermic reaction, and for the temperature increase of the gases (sensible heat), and for any heat loss to the environment; the quantity of air required is up to 10% more than that needed to react with that amount of fuel.

Referring now to FIG. 1 there is shown a reaction module 10 suitable for use as a steam reforming reactor. The reaction module 10 consists of two reactor blocks 12a and 12b each of which consists of a stack of plates that are rectangular in plan view, each plate being of corrosion resistant high-temperature alloy. Flat plates are arranged alternately with castellated plates so as to define straight-through channels between opposite ends of the stack, each channel having an active part of length 600 mm. By way of illustration, the height of the castellations (typically in the range 2-10 mm) might be 3 mm in a first example, or might be 10 mm in a second example, while the wavelength of the castellations might be such that successive ligaments are 20 mm apart in the first example or might be 3 mm apart in the second example. All the channels extend parallel to each other, there being headers so that a steam/methane mixture can be provided to a first set of channels 15 and an air/methane mixture provided to a second set of channels 16, the first and the second channels alternating in the stack (the channels 15 and 16 being represented diagrammatically), such that the top and bottom channels in the stack are both combustion channels 16. Appropriate catalysts for the respective reactions are provided on corrugated foils (not shown) in the active parts of the channels 15 and 16, so that the void fraction is about 0.9. A flame arrestor 17 is provided at the inlet of each of the combustion channels 16.

By way of example there may be over fifty such castellated plates in each stack.

The steam/methane mixture flows through the reactor blocks 12a and 12b in series, there being a duct 20 connecting the outlet from the channels 15 of the first reactor block 12a to the inlet of the channels 15 of the second reactor block 12b. Similarly the combustion mixture also flows through the reactor blocks 12a and 12b in series, there being a duct 22 connecting the outlet from the channels 16 of the first reactor block 12a to the inlet of the channels 16 of the second reactor block 12b. The duct 22 includes an inlet 24 for additional air, followed by a static mixer 25, and then an inlet 26 for additional fuel, followed by another static mixer 27.

In use of the reaction module 10, the steam/methane mixture is preheated to 620° C., and supplied to the reaction module 10 to flow through the reactor blocks 12a and 12b. A mixture of 80% of the required air and 60% of the required methane (as fuel) is preheated to 550° C., which is below the auto-ignition temperature for this composition, and is supplied to the first reactor block 12a. In both cases the preheating may be carried out by heat exchange with exhaust gases that have undergone combustion within the module 10. The temperature rises as a result of combustion at the catalyst, and the gases that result from this combustion emerge at a temperature of about 700° C. They are mixed with the remaining 20% of the required air (by the inlet 24 and the static mixer 25), and then with the remaining 40% of the required methane (by the inlet 26 and the static mixer 27), so that the gas mixture supplied to the combustion channels 16 of the second reactor block 12b is at about 600° C., which is again below the auto-ignition temperature for this mixture (which contains water vapour and carbon dioxide as a consequence of the first stage combustion). By adjusting the temperature of the additional air supplied at the inlet 24, the temperature of the resulting mixture can be controlled to be below the auto-ignition temperature.

By way of example the gas flow rates may be such that the space velocity is preferably between 14000 and 20000/hr and possibly more particularly between 15000 and 18000/hr for the steam methane reforming channels (considering the reaction module 10 as a whole), and is preferably between 19000 and 23000/hr for the combustion channels (considering the reaction module 10 as a whole).

Referring now to FIG. 2, this shows graphically the variations in temperature T along the length L of the combustion channels 16 (marked A), and that along the reforming channels 15 (marked B). The portion of the graph between L=0 and L=0.6 m corresponds to the first reactor block 12a, while the portion of the graph between L=0.6 m and L=1.2 m corresponds to the second reactor block 12b. It will be noted that the temperature T in a reforming channel 15, once combustion has commenced, is always lower than the temperature T in the adjacent combustion channel 16. The combustion gas temperature undergoes a downward step change as a result of the added air (from inlet 24) between the first reactor block 12a and the second reactor block 12b (at position L=0.6 m). The variation of conversion of methane, C, in the steam reforming reaction with length L is shown by the graph marked P. The conversion increases continuously through the reaction module 10 and reaches a value of about 80%, which is close to the equilibrium conversion under the reaction conditions.

It will be understood that adjusting the space velocities in the combustion channels and in the reforming channels, and adjusting the proportion of fuel and of air provided for combustion to each reactor block, ensures that a satisfactory temperature distribution is achieved throughout the reactor blocks, and that thermal stresses within each reactor block are minimised. This ensures that the reactor module operates within safe margins, without risk of damage to the reactor blocks. It will also be appreciated that the variations in temperature and conversion shown in FIG. 2 are by way of example only, and that the temperature distribution and consequently the conversion will be slightly different for example if the combustion catalysts are altered or if the ratio of fuel to air is altered.

It will be appreciated that the description given above is by way of example only and that many changes may be made while remaining within the scope of the present invention. For example the dimensions of the channels 15 and 16 and of the reactor blocks 12 may differ from those indicated above. The proportions of air and methane supplied to the first reactor block 12a may differ from the proportions mentioned above. The proportion of fuel provided initially may be between 50% and 65%, more preferably 55% with the remaining 35% to 50%, preferably 45%, being provided between the blocks 12a and 12b. For example 100% of the required air and 65% of the required fuel might be provided initially; and the remaining 35% of the fuel provided between the blocks 12a and 12b, although in that case it may be desirable to provide a heat exchanger (not shown) to cool the out-flowing gases to ensure the temperature is below the auto-ignition temperature. In every case the additional fuel is preferably added to a gas mixture that is below the auto-ignition temperature for the gas mixture under the prevalent conditions of gas composition and pressure. Where only part of the air is provided initially, as described above, this proportion is preferably at least 50%, and preferably no greater than 90%, more preferably between 75% and 85%, and most preferably 80% as in the example above.

It should be understood that the catalyst-carrying foils in the channels 15 and 16 preferably extend the entire length of the respective channels, apart from the initial part of the combustion channel 16 occupied by the flame arrestor 17. In a modification, no reforming catalyst is provided in an initial portion of each reforming channel 15, this initial non-catalytic portion being longer than the length of the flame arrestor 17, so that the gas mixture that is to undergo reforming is preheated before it reaches the reforming catalyst.

It should be appreciated that where the fuel gas consists of or contains a significant concentration (say >5%) of species such as H₂ and CO that have rapid combustion kinetics relative to methane, more than two reactor blocks and inter-stage mixing positions may be employed in order to control the temperature profile in the reactor module and prevent hot spots and adverse thermal gradients being generated.

The ability to modulate the proportions of fuel and air fed to each stage can also be used to compensate for reductions in catalyst activity over time. A further refinement with this arrangement is the ability to recycle some of the produced syngas to the fuel mixing stages to maintain the temperature profile in the reactor module as the combustion catalyst deactivates over time.

As will be appreciated, steam methane reforming may form part of a process for converting methane to longer-chain hydrocarbons, the synthesis gas produced by reforming then being subjected to Fischer-Tropsch synthesis. Alternatively, the synthesis gas may be subjected to a catalytic process to form methanol. The steam methane reforming in any such plant may be carried out using one or more reaction modules 10 as described above. A preferred plant incorporates several such reaction modules arranged in parallel, so that the plant capacity can be adjusted by changing the number of reaction modules that are utilised.

In the reaction module 10 shown in FIG. 1, and considering only the combustion channels 16, a platinum-palladium catalyst may be provided in both reactor blocks 12a and 12b. Alternatively the catalyst may be different in the two reactor blocks 12a and 12b. For example the catalyst in the first reactor block 12a may be platinum-palladium, and the catalyst in the second reactor block 12b instead might be platinum only. It will be appreciated that the oxygen partial pressure within the second reactor block 12b is less than that in the first reactor block 12a because of the combustion that has taken place. If a platinum-palladium catalyst is used in the second reactor block 12b a problem can arise, because this low oxygen partial pressure encourages the transformation of palladium oxide to palladium metal, and palladium metal is less effective as a combustion catalyst than palladium oxide. Hence there can be a benefit from using a platinum-only catalyst within the second reactor block 12b, or from using a platinum-palladium mixture with a high proportion of platinum in the second reactor block 12b. Platinum is catalytically active in the metal form, rather than the oxide form, and therefore the activity of the catalyst is not adversely affected by the low oxygen partial pressure within the second reactor block 12b. As another alternative a platinum-only catalyst could be used in both reactor blocks 12a and 12b. However, a platinum catalyst has a lower light-off temperature than a platinum-palladium catalyst, so it is not as suitable for use in the first reactor block 12a, and in addition, the oxygen partial pressure is higher in the first reactor block 12a and therefore the platinum-only catalyst does not provide the benefit that it would in the second reactor block 12b.

An alternative reaction module 100 is shown in FIG. 5, to which reference is now made, components that are the same as those of the module 10 being referred to by the same references. The reaction module 100 consists of two reactor blocks 12a and 12b represented schematically, and the steam/methane mixture flows through the reactor blocks 12a, 12b in series via the duct 20 as described above. Separate combustion mixtures are supplied to each of the reactor blocks 12a and 12b, and the exhaust gases emerging from the combustion channels 16 of both the reactor blocks 12a and 12b are provided to a common exhaust vent 102 (or to two separate exhaust vents). The combustion mixture supplied to the second reactor block 12b is preheated to 550° C., which is below its auto-ignition temperature, by preheating the air and fuel in heat exchangers 104 and 105 heated by the exhaust gases in the vent 102, the preheated air and fuel then being mixed in a mixer 27. (The combustion mixture supplied to the first reactor block 12a may be preheated similarly.)

The combustion mixture supplied to the first reactor block 12a of the module 100 may have the same composition as is supplied to the second reactor block 12b. Hence 50% of the total fuel requirement may be supplied to the first reactor block 12a and the remaining 50% to the second reactor block 12b, each block being provided with the same volume of air. However, it should be noted that the volume of fuel supplied to the first reactor block 12a may be the same as that supplied to the first reactor block of the module 10. Consequently the overall amount of fuel supplied to the module 100 may be greater than the amount of fuel supplied to the module 10.

Alternatively a somewhat higher proportion of the total fuel requirement may be provided to the first reactor block 12a, for example 55%, and the remaining 45% of the total being provided to the second reactor block 12b. By venting at least part of the exhaust gases from the first stage combustion reaction, the percentage of the product gases water vapour and carbon dioxide in the channels of the second reactor block 12b is reduced compared to that in FIG. 1. This, in turn, contributes to an increased partial oxygen pressure in the second reactor block 12b. Consequently a palladium/platinum catalyst is suitable for use in the combustion channels 16 of both the reactor blocks 12a and 12b. The temperature distribution through the module 100 is substantially the same as that described in relation to FIG. 2 in the module 10, and the overall conversion achieved in the steam methane reforming channels is substantially the same.

Referring now to FIG. 3 this shows a flow diagram of a system 30 for supplying a steam methane mixture to a reforming module 10 as described above, or to a reforming module 100 as described above, as part of a plant for processing natural gas. The processing plant, in this example, converts natural gas to longer chain hydrocarbon products. The natural gas is initially conditioned to remove impurities such as mercury or sulphur and so provide a feed stream of clean natural gas, typically about 90% of methane with small percentages of other alkanes. This is used to generate synthesis gas, by steam methane reforming. The synthesis gas is subjected to Fischer-Tropsch synthesis to generate the longer chain hydrocarbons, leaving a residual tail gas; this tail gas may consist primarily of short chain alkanes, carbon monoxide, carbon dioxide, water vapour, and hydrogen.

The system 30 is intended for use in such a processing plant, and in this example is provided with three input streams: the feed stream 31 of clean natural gas, a supply of steam 32, and tail gas 33 recycled from the Fischer-Tropsch synthesis plant. The system 30 generates a mixture containing natural gas and steam, and subjects this to pre-reforming, for example using a nickel catalyst, in a pre-reformer 35, to convert any C2₊ hydrocarbons (ethane, propane, etc.) to methane, carbon monoxide and hydrogen. The flows are ideally such that the steam:methane molar ratio after pre-reforming is between 1.4 and 1.6 to 1. The resulting gas mixture 36 consists primarily of methane and steam, and is supplied to one or more reforming reactor modules 10 as described above.

The system 30 includes a control system 38 to control the ratio of steam to carbon (whether in methane or another alkane) that is supplied to the pre-reformer 35. During normal operation the steam:carbon ratio will be about 1.4 to 1, but during start-up a higher proportion of steam is used to avoid coking of the catalyst in the reformer module 10 while the catalyst temperatures rise to their target values. Flow transmitters 40 measure the flow of the input streams 31, 32 and 33, and supply data to a fuel flow controller 42. The fuel flow controller 42 operates a control valve 44 to adjust the flow rate of the steam and so to ensure the required steam to carbon ratio. Signals from the flow transmitter 40 measuring the feed gas flow 31 are also transmitted to a flow controller 46 that operates a vent valve 48 to divert any peaks in the feed gas flow rate out of the system 30, for example to a flare (not shown).

A heat exchanger 50 is provided to heat the recycled tail gas stream 33 to the same temperature as the steam 32 and feed gas 31, which in this plant have been previously heated to an elevated temperature. The gas streams 31, 32 and 33 are then mixed, and the resulting gas mixture is then further heated by a pre-heater 52 to the required input temperature for the pre-reformer 35, typically about 425° C.

The flow rates of the feed gas 31 and of the tail gas 33 as measured by the corresponding flow transmitters 40, but allowing for the effect of the vent valve 48, are calculated and transmitted at 54 for controlling the steam methane reforming module 10 (as described below).

The reaction in the pre-reformer 35 may be catalysed by a pre-reduced and stabilised nickel based catalyst. Because the tail gas 33 is included within the gas mixture, the gas mixture contains carbon monoxide and carbon dioxide, and consequently the reaction in the pre-reformer is slightly exothermic, and the temperature of the resulting output stream 36 is about 540° C.

Controlling the temperature and composition of the mixture fed to the pre-reformer 35 is necessary in order to protect the catalysts in the pre-reformer 35 and the reforming reactor module 10. For example, steam should not be introduced if condensing conditions are present, for example if the temperature within the pre-reformer 35 is less than 180° C. Steam also must not be allowed to flow through the pre-reformer 35 on its own for longer than 15 minutes, or the catalyst may start to undergo an irreversible oxidation reaction. To prevent oxidation of the catalyst, the steam 32 should be mixed with at least a small proportion of hydrogen or natural gas, for example 10 mole %. The pre-reformer 35 can pass natural gas at up to 200° C. without detriment, but the catalyst will be destroyed by coking within about 20 s if natural gas is passed over the catalyst at above 250° C. It is therefore important to shut off the natural gas feed stream 31 if the steam supply 32 ceases, and the tail gas stream 33 must also be shut off. The pre-reformer 35 must not be de-pressurised faster than 1 bar/min to avoid damaging the catalyst, and should also not be heated or cooled faster than 1° C./min.

Referring now to FIG. 4 there is shown a flow diagram of a system 60 to control operation of a steam reforming module 10 as shown in FIG. 1. The gas supplies in this case are: desulphurised natural gas 61 as fuel; the gas mixture 36 from the pre-reformer 35; and blown air 62. The gas mixture 36, which consists primarily of steam and methane, is subjected to a control loop comprising a pressure transmitter 64 that provides data about the pressure of the gas mixture 36 to a pressure controller 65; the pressure controller 65 can adjust the flow rate using a control valve 66 and can open a vent valve 67 to divert the gas mixture to a flare if the pressure of the gas mixture 36 exceeds a predetermined safe threshold pressure for the reactor module 10. The gas mixture 36 is then fed through a pre-heater 68 into the reactor module 10.

The reactor module 10 is also supplied with a mixture of blown air 62 and desulphurised natural gas 61 for the combustion reactions. The blown air 62 is first heated through a pre-heater 604, and then its temperature is measured by a temperature sensor 605. The flow rate of air supplied to the module 10 is adjusted by a valve 606 in response to control signals from a controller 70. The controller 70 receives data from both the temperature sensor 605 and also an oxygen sensor 607 at the outlet for the combustion gases from the second module 12b.

The blown air 62, after passing through the valve 606, is separated into a first air flow supplied through a heat exchanger 610 to a static mixer 618 (to be mixed with fuel gas) at the inlet for the first reactor module 12a, and a second air flow supplied through a heat exchanger 611 to the inlet 24 of the static mixer 25 at the outlet from the first reactor module 12a. The ratio of the first and second air flows is controlled by a valve 608 in the second air flow. This valve 608 is controlled by a controller 72 that receives input signals from a temperature sensor 609 at the outlet from the static mixer 25, and a flow sensor 74 at the inlet to the valve 608. The heat exchangers 610 and 611 can be controlled separately, the heat exchanger 610 heating the air to a temperature of around 500° C., whilst the second stage heater 611 heats the air to a temperature in the region of 300° C.

The desulphurised gas 61 that is the fuel for combustion is controlled in a similar manner to the blown air 62, although as explained above the mixture supplied to the first reactor block 12a may be 80% of the required air and 55% or 60% of the required fuel. The rest of the required air and the rest of the required fuel are introduced through the static mixers 25 and 27 between the first reactor block 12a and the second reactor block 12b. The fuel flow 61 is split into two flows: a first flow via a control valve 614 and a heat exchanger 616 to the static mixer 618 at the inlet to the first reactor block 12a, and a second flow via a control valve 615 and a heat exchanger 617 to the inlet 26 of the static mixer 27. The first flow is heated to about 500° C. or 550° C. by the heat exchanger 616, whereas the second flow is heated to about 300° C. by the heat exchanger 617.

The overall control of the system 60 is provided by a controller 612. The controller 612 receives the signals 54 indicating the flows of natural gas 31 and tail gas 33 (see FIG. 3) from which it can deduce the flow of methane to be reformed. The controller 612 also receives data from a temperature sensor 613 at the outlet of the second reactor block 12b. It also receives data from the controller 70 about the flow of blown air 62. The controller 612 controls the flow of fuel through the valves 614 and 615 by providing signals to respective valve controllers 76 and 78 that also receive data on the flow rate from flow sensors 77 and 79. The controller 612 also controls the flow rate of blown air 62 through the valve 606, by providing control signals to the controller 70.

Thus in operation of the system 60, the air supply to the module 10, that is to say the flow of the blown air 62, is controlled by the controller 612 and the controller 70 in accordance with the quantity of methane to be reformed. If the oxygen level sensed by the sensor 607 at the outlet from the module 10 decreases, then the valve 606 is adjusted to increase the flow of blown air 62 to the module 10. If the oxygen level increases, then the flow of blown air 62 to the module 10 is decreased, and the flow rate of the fuel 61 is also reduced in proportion.

The flow rate of the fuel 61 is also controlled in accordance with the quantity of methane to be reformed. In addition, if the temperature sensed by the sensor 613 at the outlet from the module 10 becomes excessively high, then the flow rate of the fuel 61 to both the reactor blocks 12a and 12b would be reduced. On the other hand, if the temperature sensed by the sensor 609 at the outlet from the static mixer 25 rises, the air supply to the inlet 24 of the static mixer 25 is increased (or alternatively the heat exchanger 611 might be adjusted to achieve a lower temperature). This ensures that the gas mixture in the mixer 27 is below its auto-ignition temperature.

A system to control operation of a steam reforming module 100 as shown in FIG. 5 may be similar to the system 60 described above with the exception that the output of the combustion channels from the first reactor block 12a is vented, and a new mixture of air and fuel is supplied. There is therefore no need for the static mixer 25, only the mixer 27. In the module 100 the temperatures and quantities of gas input into the two stages can be independently controlled, and the temperature of the air and fuel for the second reactor block 12b, controlled by heat exchangers 611, 617 (which correspond to the heat exchangers 105 and 104 of FIG. 5) respectively, can be up to 500° C. or 550° C. (rather than 300° C. as described above).

The control system 30 is described as receiving two sources of hydrocarbons: natural gas 31 and a tail gas 33. It will be appreciated that this is by way of example only, as the requirement is only that there must be at least one gas supply that contains hydrocarbons, typically a natural gas supply. If a second source of gaseous hydrocarbons is available, then this may also be supplied in an analogous way to the tail gas 33. For example, where such a pre-reformer 35 and associated control system 30 are provided in the context of a different processing plant, for example a processing plant for producing methanol rather than for producing longer chain hydrocarbons, then there may be only a single such gas supply to the pre-reformer 35, or there may also be a tail gas of a different composition from that described above.

1. A catalytic reaction module for performing an endothermic reaction, the module comprising a plurality of separate reactor blocks, each reactor block defining a multiplicity of first and second flow channels arranged alternately within the block to ensure thermal contact between the first and second flow channels, the reactor blocks being arranged and connected for series flow of a gas mixture to undergo the endothermic reaction in the first flow channels and also for flow of a combustible gas mixture in the second flow channels to undergo combustion, such that the endothermic reaction mixture flows in series through the reactor blocks, wherein between successive reactor blocks, the module comprises means to introduce additional oxygen-containing gas and means to introduce additional fuel into an outflowing gas mixture from the second flow channels that results from combustion.

2. (canceled)

3. (canceled)

4. A reaction module as claimed in claim 1 wherein the module is arranged such that the combustible gas mixture provided to a reactor block is at an elevated temperature below its auto-ignition temperature, the temperature being raised at least in part as a result of combustion of combustible gas mixture in one or more of the reactor blocks.

5. A reaction module as claimed in claim 4 arranged such that the combustible gas mixture provided to each reactor block in the module is at a said elevated temperature.

6. (canceled)

7. A reaction module as claimed in claim 1 wherein, within a reactor block, the first flow channels and the second flow channels extend in parallel directions, and the combustible gas mixture and the endothermic reaction mixture flow in the same direction.

8. A reaction module as claimed in claim 1 wherein the flow channels within each reactor block are of length at least 300 mm, more preferably at least 500 mm, but preferably no longer than 1000 mm.

9. A reaction module as claimed in claim 1 wherein a flame arrestor is provided at the inlet to each second flow channel.

10. A method of performing an endothermic reaction, using a reaction module as claimed in claim 1 wherein the heat required for the endothermic reaction is provided by a combustion reaction in an adjacent channel to the endothermic reaction, and wherein the endothermic reaction is carried out in a plurality of successive stages, and the combustion reaction is carried out in at least two stages in sequence, in the same sequence as the endothermic reaction, with treatment of the combustion gas mixture emerging from one stage before it is introduced to the next stage.

11. (canceled)

12. (canceled)

13. A method as claimed in claim 10 wherein the treatment comprises changing its temperature and adding additional fuel.

14. A method as claimed in claim 13 wherein the temperature is changed by adding a gas or vapour.

15. A control system for a catalytic reaction module for performing an endothermic reaction, the module comprising a plurality of separate reactor blocks, each reactor block defining first and second flow channels, the reactor blocks being arranged and connected for series flow of a gas mixture to undergo the endothermic reaction in the first flow channels of the reactor blocks, and for flow of a combustible gas mixture in the second flow channels, such that the combustible gas mixture flows through the reactor blocks and that the endothermic reaction mixture flows in series through the reactor blocks, wherein the control system comprises means to monitor the flow rate of the mixture that is to undergo the endothermic reaction, and means to control the flow rate of the mixture that is to undergo combustion in accordance with the monitored flow rate wherein the flow rate control means is arranged to provide to the first reactor block a proportion of between 50% and 70% of the fuel supplied to the module.

16. (canceled)
